(19) United States
(12) Patent Application Publication
(10) Pub. No.: US 2022/0008378 A1
(43) Pub. Date: Jan. 13, 2022

(54) Formulations and methods

(71) Applicants: JONATHAN TESSMAR-BELL, Scottsdale, AZ (US); BYRON JOEL CURRIER, Mesa, AZ (US)

(72) Inventors: JONATHAN TESSMAR-BELL, Scottsdale, AZ (US); BYRON JOEL CURRIER, Mesa, AZ (US)

(21) Appl. No.: 17/300,457

(22) Filed: Jul. 6, 2021

Related U.S. Application Data
(60) Provisional application No. 63/102,919, filed on Jul. 9, 2020.
(60) Provisional application No. 63/102,910, filed on Jul. 7, 2020.

(51) Int. Cl. A61K 31/352; A61K 31/05; A61K 9/107; A61K 47/36; A61K 47/14; A61K 31/4045

(52) U.S. Cl. CPC A61K 31/352; A61K 31/05; A61K 31/4045; A61K 47/36; A61K 47/14; A61K 9/1075

(57) ABSTRACT

The present invention generally relates to formulations of and methods using one or more cannabis-derived compounds. In one aspect, the present invention provides a nanoemulsion. In addition to one or more cannabis-derived compounds (e.g., THC and/or CBD), the nanoemulsion typically includes at least one surfactant (e.g., Quillaja saponis), at least one encapsulator (e.g., modified food starch and/or maltodextrin), and at least one carrier (e.g., MCT coconut oil). Other suitable components such as one or more sweeteners (e.g., Xylitol) and/or one or more flow aids (e.g., Hydrophilic Fumed Silica having a Specific Surface Area (BET) between 175 m²/g and 225 m²/g) may be included. In certain cases, the nanoemulsion includes non-cannabis derived compounds such as caffeine and melatonin. The nanoemulsion typically has a Particle Size Distribution D50 of less than 500 nm, less than 400 nm, less than 300 nm, less than 200 nm or less than 100 nm.

### FIELD OF THE INVENTION

The present invention generally relates to formulations of and methods using one or more cannabis-derived compounds.

### BACKGROUND OF THE INVENTION

There have been reports of formulations of and methods using cannabis-derived compounds. For instance, U.S. Pat. No. 10,617,636, entitled “Cannabis infused sweeteners and elixirs” allegedly reports the following: “A cannabis infused sweetener comprising cannabis oil; at least one emulsifying agent selected from the group consisting of xanthan gum, guar gum, lecithin, carrageen, monoglycerides, natural emulsifiers and organic emulsifiers that are safe for ingestion by humans; and a sweetener; and a cannabis infused elixir comprising cannabis oil; at least one emulsifying agent; a sweetener and flavoring extracts or fruit syrups; both the sweeteners and the elixirs are bio-available, fast acting and highly metabolizable.” Abstract.

U.S. Pat. No. 10,568,920, entitled “Pharmaceutical compositions containing cannabis, uses thereof and methods for improving energy levels and/or alleviating fatigue” allegedly discusses the following: “A pharmaceutical composition containing cannabis for increasing energy levels and/or alleviating fatigue in a patient. Also provided are a use of the pharmaceutical composition for increasing energy levels and/or alleviating fatigue in a patient, and methods for increasing energy levels and/or alleviating fatigue. The pharmaceutical composition preferably includes a combination of therapeutically effective amounts of one or more of the following medicinal ingredients: cannabis, an herb, vitamins, and/or fructose.” Abstract.

U.S. Pat. No. 10,517,848, entitled “Cannabis-based bioactive formulations and methods for use thereof” allegedly reports the following: “Disclosed are compositions and methods for mitigating a tolerance effect from ingestion of cannabis-based medicines in human subjects, for example, subjects having an increased risk of unresponsiveness to one or more therapeutic agents such as, for example, antidepressants and antianxiety drugs. In some methods, the human subjects have been previously treated with one or more therapeutic agents such as, for example, antidepressants and antianxiety drugs, and have developed at least partial tolerance to the one or more therapeutic agents.” Abstract.

Despite the reports, there is still a need in the art for novel formulations of and methods using cannabis-derived compounds.

### SUMMARY OF THE INVENTION

In one aspect, the present invention provides a nanoemulsion embodiment. The nanoemulsion comprises: a surfactant (e.g., Quillaja saponins), medium-chain triglycerides (e.g., MCT coconut oil), and 1-50 mg of CBD or THC. The nanoemulsion has a Particle Size Distribution D50 of less than 500 nm, less than 400 nm, less than 300 nm, less than 200 nm or less than 100 nm.

In one aspect, the present invention provides a nanoemulsion embodiment. The nanoemulsion comprises: a surfactant (e.g., Quillaja saponins), medium-chain triglycerides (e.g., MCT coconut oil), and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion comprises: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol) and 1-50 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion comprises: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol) and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion comprises: a surfactant (e.g., Quillaja saponins), medium-chain triglycerides (e.g., MCT coconut oil), 10 mg to 250 mg caffeine, and 1-50 mg of CBD or THC.

In another aspect, the present invention provides another nanoemulsion embodiment. The nanoemulsion comprises: a surfactant (e.g., Quillaja saponins), medium-chain triglycerides (e.g., MCT coconut oil), 10 mg to 250 mg caffeine, and 1-10 mg of CBD or THC.

In another aspect, the present invention provides another nanoemulsion embodiment. The nanoemulsion comprises: a surfactant (e.g., Quillaja saponins), medium-chain triglycerides (e.g., MCT coconut oil), 1 mg to 10 mg melatonin, and 1-50 mg of CBD or THC.

In another aspect, the present invention provides another nanoemulsion embodiment. The nanoemulsion comprises: a surfactant (e.g., Quillaja saponins), medium-chain triglycerides (e.g., MCT coconut oil), 1 mg to 10 mg melatonin, and 1-10 mg of CBD or THC.

In another aspect, the present invention provides another nanoemulsion embodiment. The nanoemulsion comprises: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 10 mg to 250 mg caffeine and 1-50 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion comprises: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 10 mg to 250 mg caffeine and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion comprises: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 1 mg to 10 mg melatonin and 1-50 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion comprises: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 1 mg to 10 mg melatonin and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion consists essentially of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), and 1-50 mg of CBD or THC. The nanoemulsion has a Particle Size Distribution D50 of less than 500 nm, less than 400 nm, less than 300 nm, less than 200 nm or less than 100 nm. The transitional phrase “consists essentially of” in the context of this embodiment means that the nanoemulsion does not include any cannabis-derived compound except for CBD or THC; it may include other sweeteners, flavorants or buffers.

In another aspect, the present invention provides another nanoemulsion embodiment. The nanoemulsion consists essentially of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), and 1-10 mg of CBD or THC. The nanoemulsion has a Particle Size Distribution D50 of less than 500 nm, less than 400 nm, less than 300 nm, less than 200 nm or less than 100 nm. The transitional phrase “consists essentially of” in the context of this embodiment means that the nanoemulsion does not include any cannabis-derived compound except for CBD or THC; it may include other sweeteners, flavorants or buffers.

In another aspect, the present invention provides another nanoemulsion embodiment. The nanoemulsion consists essentially of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 10 mg to 250 mg caffeine and 1-50 mg of CBD or THC. The nanoemulsion has a Particle Size Distribution D50 of less than 500 nm, less than 400 nm, less than 300 nm, less than 200 nm or less than 100 nm. The transitional phrase “consists essentially of” in the context of this embodiment means that the nanoemulsion does not include any cannabis-derived compound except for CBD or THC; it may include other sweeteners, flavorants or buffers.

In another aspect, the present invention provides another nanoemulsion embodiment. The nanoemulsion consists essentially of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 10 mg to 250 mg caffeine and 1-10 mg of CBD or THC. The nanoemulsion has a Particle Size Distribution D50 of less than 500 nm, less than 400 nm, less than 300 nm, less than 200 nm or less than 100 nm. The transitional phrase “consists essentially of” in the context of this embodiment means that the nanoemulsion does not include any cannabis-derived compound except for CBD or THC; it may include other sweeteners, flavorants or buffers.

In another aspect, the present invention provides another nanoemulsion embodiment. The nanoemulsion consists essentially of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 1 mg to 10 mg melatonin and 1-50 mg of CBD or THC. The nanoemulsion has a Particle Size Distribution D50 of less than 500 nm, less than 400 nm, less than 300 nm, less than 200 nm or less than 100 nm. The transitional phrase “consists essentially of” in the context of this embodiment means that the nanoemulsion does not include any cannabis-derived compound except for CBD or THC; it may include other sweeteners, flavorants or buffers.

In another aspect, the present invention provides another nanoemulsion embodiment. The nanoemulsion consists essentially of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 1 mg to 10 mg melatonin and 1-10 mg of CBD or THC. The nanoemulsion has a Particle Size Distribution D50 of less than 500 nm, less than 400 nm, less than 300 nm, less than 200 nm or less than 100 nm. The transitional phrase “consists essentially of” in the context of this embodiment means that the nanoemulsion does not include any cannabis-derived compound except for CBD or THC; it may include other sweeteners, flavorants or buffers.

In another aspect, the present invention provides another nanoemulsion embodiment. The nanoemulsion consists of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), and 1-50 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion consists of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion consists of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 10 mg to 250 mg caffeine and 1-50 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion consists of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 10 mg to 250 mg caffeine and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion consists of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 1 mg to 10 mg melatonin and 1-50 mg of CBD or THC. The nanoemulsion has a Particle Size Distribution D50 of less than 500 nm, less than 400 nm, less than 300 nm, less than 200 nm or less than 100 nm.

In another aspect, the present invention provides another nanoemulsion embodiment. The nanoemulsion consists of: a surfactant (e.g., Quillaja saponins), modified food starch and/or maltodextrin, medium-chain triglycerides (e.g., MCT coconut oil), a flow aid (e.g., Hydrophilic Fumed Silica having a specific surface area ranging from 175 m²/g to 225 m²/g), sweetener (e.g., xylitol), 1 mg to 10 mg melatonin and 1-10 mg of CBD or THC. The nanoemulsion has a Particle Size Distribution D50 of less than 500 nm, less than 400 nm, less than 300 nm, less than 200 nm or less than 100 nm.

In another aspect, the present invention provides a method of producing a sense of well-being in a person. The method comprises ingestion of a nanoemulsion according to the preceding paragraphs.

In another aspect, the present invention provides a method of reducing anxiety in a person. The method comprises ingestion of a nanoemulsion according to the preceding paragraphs.

In another aspect, the present invention provides a method of aiding a person to fall asleep. The method comprises ingestion of a nanoemulsion according to the preceding paragraphs.

### DETAILED DESCRIPTION

“Surfactant” refers to a compound that lowers the surface tension between two liquids, between a gas and a liquid, or between a liquid and a solid. Nonlimiting examples of surfactants include: Quillaja saponins; TWEEN® 80; polyethylene glycol octyl phenyl ether; polyoxyethylene sorbitan monostearate; polyethylene glycol hydrogenated castor oil; sodium lauryl sulfate; and poloxamer 407.

“Encapsulator” refers to a compound that facilitates confinement of one molecule within a larger molecule or collection of molecules. Nonlimiting examples of encapsulators include: modified food starch, unmodified starch, and maltodextrose and combinations thereof.

“Carrier” refers to a compound that encapsulates another compound in an emulsion. Non-limiting examples of carriers include: long-chain triglycerides; and, medium-chain triglycerides.

“Anti-oxidant” refers to a compound that inhibits oxidation. Non-limiting examples of anti-oxidants include: vitamin E; vitamin C; beta-carotene; lycopene; lutein; and, zeaxanthin.

“Sweetener” refers to a compound that provides a sweet taste in food and beverages. Non-limiting examples of sweeteners include: xylitol; acesulfame potassium; aspartame; mogrosides; saccharin; stevia; sucralose; and natural sweeteners (e.g., isolated from plants not synthesized in a laboratory).

“THC” refers to tetrahydrocannabinol. One non-limiting composition including THC is a distillate that contains at least 85 percent THC.

“CBD” refers to cannabidiol. One non-limiting composition including CBD is a distillate that contains at least 85 percent CBD.

“Particle Size Distribution D50” refers to the median diameter or the medium value of the particle size distribution. It is the value of the particle diameter at 50% in the cumulative distribution.

“TWEEN® 80” refers to a polyethylene sorbitol ester, otherwise known as Polysorbate 80 or polyoxyethylene sorbitan monooleate. It has a molecular weight of 1.31 kDa and works as a stabilizer and emulsifier.

“Modified starch” refers to a starch derivative prepared by physically, enzymatically or chemically treating native starch to change its properties.

“Flow aid” refers to a compound or composition that improves the flow properties of a formulation. One example of a flow aid is Hydrophilic Fumed Silica having a Specific Surface Area (BET) between 175 m²/g and 225 m²/g.

The present invention provides formulations including cannabis-derived compounds such as THC and CBD (e.g., 1-50 mg). In addition to one or more cannabis-derived compounds, the formulations typically include at least one surfactant (e.g., Quillaja saponis), at least one encapsulator (e.g., modified food starch and/or maltodextrin), and at least one carrier (e.g., MCT coconut oil). Other suitable components such as one or more sweeteners (e.g., Xylitol) and/or one or more flow aids (e.g., Hydrophilic Fumed Silica having a Specific Surface Area (BET) between 175 m²/g and 225 m²/g) may be included. In certain cases, the formulations include non-cannabis derived compounds such as caffeine and melatonin. The formulation is typically in the form of a nanoemulsion having a Particle Size Distribution D50 of less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 1 to 10 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 10 to 50 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 20 to 30 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 1 mg to 10 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 10 mg to 50 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 20 mg to 30 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 1 to 10 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 10 to 50 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 20 to 30 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 1 to 5 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 1 to 5 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 1 to 5 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 5 to 9 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 5 to 9 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 5 to 9 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 1 to 10 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 10 to 50 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 20 to 30 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 1 to 10 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 10 to 50 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 20 to 30 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 1 to 10 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 10 to 50 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 20 to 30 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 1 to 5 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 1 to 5 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 1 to 5 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 5 to 9 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 5 to 9 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 5 to 9 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 1 to 10 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 10 to 50 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 20 to 30 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 1 to 10 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 10 to 50 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 20 to 30 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 1 to 10 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 10 to 50 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 20 to 30 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 1 to 5 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 1 to 5 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 1 to 5 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g and 5 to 9 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 10 mg to 250 mg caffeine, and 5 to 9 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 10 mg melatonin, and 5 to 9 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g., 150 mg), and 1 mg to 10 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g., 150 mg), and 10 mg to 50 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g., 150 mg), and 20 mg to 30 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 1 to 10 mg of CBD. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g., 150 mg), and 1 to 10 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g., 150 mg), and 10 to 50 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g., 150 mg), and 20 to 30 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 1 to 10 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 10 to 50 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 20 to 30 mg of THC. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g. 150 mg), and 1 to 10 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g. 150 mg), and 10 to 50 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 100 mg to 200 mg caffeine (e.g. 150 mg), and 20 to 30 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 1 to 10 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 10 to 50 mg of a CBD/THC mixture. It is a nanoemulsion having a Particle Size Distribution D50 of less than less than 500 nm, less than 400 nm, less than 300 nm, less than 200 nm or less than 100 nm.

In one embodiment, the formulation of the present invention comprises Quillaja saponins, modified food starch and/or maltodextrin, medium-chain triglycerides, xylitol or other sweetener, Hydrophilic Fumed Silica having a specific surface area ranging from 175 m²/g to 225 m²/g, 1 mg to 5 mg melatonin (e.g., 3 mg), and 20 to 30 mg of a CBD/THC mixture. It is a nanoemulsion having a Particle Size Distribution D50 of less than less than 500 nm, less than 400 nm, less than 300 nm, less than 200 nm or less than 100 nm.

A formulation of the present invention is oftentimes made in the following way: An oil phase was sonicated first (Quillaja, Active (e.g., THC and/or CBD), water, MCT) for 1.5 hrs in an ice bath to decrease particle size. Modified food starch and water were combined in a separate beaker and an immersion blender was used to homogenize. [The MFS (Modified Food Starch) mixture was moved outside of sonication due to the breakdown of the MFS properties which created a material that was not able to be used in the fluidized bed. It was noted that water would not adhere to the MFS particles at all.] Both phases were combined and homogenized with an immersion blender. The resulting liquid nano-emulsion was then pumped into the spray drier. Spray dried powder was then fed into a fluidized bed for granulation. Granulation output of the fluidized bed drying process was then sieved at 355 micron. Granulated xylitol is added to the granulation output from the fluidized bed drying, providing the final product. In an alternate process, a microfluidizer can be used to homogenize the various mixtures.

Formulations of the present invention may be used in a variety of ways. For instance, the formulation may be added to a liquid (e.g., water), a food (e.g., pizza) or a recipe or baking (e.g., brownie batter). A person may drink the liquid or eat the food to which the formulation has been added. The person may consume the liquid or food, for example, seeking a general sense of well-being, to reduce anxiety, to aid in falling asleep, to increase quality of sleep or for other suitable reasons.

The general sense of well-being after consuming a formulation according to the present invention is typically at least 10 percent better than before consumption. In certain cases, it is 20 percent better, 30 percent better, 40 percent better or 50 percent better. Well-being can be measured using a variety of scales known to those of skill in the art, including: Satisfaction With Life Scale (SWLS), see Diener et al. in Journal of Personality Assessment; Scale of Positive and Negative Experience (SPANE); and Flourishing Scale (FS), sometimes called the Psychological Well-Being scale.

Anxiety reduction after consuming a formulation according to the present invention is typically at least 10 percent less anxious than before consumption. In certain cases, anxiety is reduced by at least 20 percent, at least 30 percent, at least 40 percent or at least 50 percent. Anxiety can be measured using a variety of scales known to those of skill in the art, including: State Trait Anxiety Inventory (STAI); Hospital Anxiety and Depression Scale (HADS); Generalized Anxiety Disorder-7 (GAD-7); and the Zung Anxiety Scale.

Sleep quality after consuming a formulation according to the present invention is typically at least 10 percent better than before consumption. In certain cases, it is 20 percent better, 30 percent better, 40 percent better or 50 percent better. Sleep quality can be measured using a variety of scales known to those of skill in the art, including: Sleep Over the Past Week Questionnaire, see SLEEP, Vol. 31, No. 3, 2008.

The formulations of the present invention provide for quick onset of action. Typically, someone who consumes such a formulation begins to feel effects (e.g., sense of well-being) in less than 20 minutes. In certain cases, the person begins to feel effects in less than 18 minutes, less than 15 minutes or even in less than 10 minutes.

In one case, a formulation embodiment of the present invention was ingested in twenty-five separate tests. Time to effect ranged from 5 minutes to 45 minutes, with an average of 17.32 minutes.

1. A nanoemulsion, wherein the nanoemulsion comprises: a surfactant, modified food starch, medium-chain triglycerides, a flow aid, a sweetener and 1-10 mg of CBD or THC, wherein the nanoemulsion has a Particle Size Distribution D50 of less than 500 nm.

2. A nanoemulsion, wherein the nanoemulsion comprises: a cannabis-derived compound, a surfactant, an encapsulator and a carrier, wherein the nanoemulsion has a Particle Size Distribution D50 of less than 500 nm.

3. The nanoemulsion according to claim 2, wherein the cannabis-derived compound is CBD or THC included in an amount ranging from 1 mg to 10 mg.

4. The nanoemulsion according to claim 2, wherein the surfactant is Quillaja saponis.

5. The nanoemulsion according to claim 2, wherein the encapsulator is modified food starch, maltodextrin or a combination of the two.

6. The nanoemulsion according to claim 2, wherein the carrier is MCT coconut oil.

7. The nanoemulsion according to claim 3, wherein the cannabis-derived compound is CBD or THC included in an amount ranging from 1 mg to 10 mg, and wherein the surfactant is Quillaja saponis.

8. The nanoemulsion according to claim 3, wherein the encapsulator is modified food starch, maltodextrin or a combination of the two, and wherein the carrier is MCT coconut oil.

9. The nanoemulsion according to claim 7, wherein the encapsulator is modified food starch, maltodextrin or a combination of the two, and wherein the carrier is MCT coconut oil.

10. A method of producing a sense of well-being in a person, wherein the method comprises ingestion of a nanoemulsion, wherein the nanoemulsion comprises: a cannabis-derived compound, a surfactant, an encapsulator, a carrier, and caffeine, wherein the nanoemulsion has a Particle Size Distribution D50 of less than 500 nm.

11. The method according to claim 10, wherein the cannabis-derived compound is CBD or THC included in an amount ranging from 1 mg to 10 mg.

12. The method according to claim 10, wherein the surfactant is Quillaja saponis.

13. The method according to claim 10, wherein the encapsulator is modified food starch, maltodextrin or a combination of the two.

14. The method according to claim 10, wherein the carrier is MCT coconut oil.

15. A method of increasing the quality of a person's sleep, wherein the method comprises the ingestion of a nanoemulstion, wherein the nanoemulsion comprises: wherein the nanoemulsion comprises: a cannabis-derived compound, a surfactant, an encapsulator, a carrier, and melatonin, wherein the nanoemulsion has a Particle Size Distribution D50 of less than 500 nm.

16. The method according to claim 15, wherein the cannabis-derived compound is CBD or THC included in an amount ranging from 1 mg to 10 mg.

17. The method according to claim 15, wherein the surfactant is Quillaja saponis.

18. The method according to claim 15, wherein the encapsulator is modified food starch, maltodextrin or a combination of the two.

19. The method according to claim 15, wherein the carrier is MCT coconut oil.
